(19) United States
(12) Patent Application Publication
(10) Pub. No.: US 2007/0169287 A1
(43) Pub. Date: Jul. 26, 2007

(54) P-diaminobenzene derivatives and dyes containing these compounds

(75) Inventors: Laurent Chassot, Praroman (CH); Hans-Jurgen Braun, Ueberstorf (CH)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/524,129

(22) Filed: Sep. 20, 2006

Related U.S. Application Data
(63) Continuation of application No. PCT/EP05/07045, filed on Jun. 30, 2005.

(30) Foreign Application Priority Data
Sep. 4, 2004 (DE) 102004042850.6

(51) Int. Cl. A61K 8/00

(52) U.S. Cl. 8/405

(57) ABSTRACT

p-Diaminobenzene derivatives of the general Formula (I) or their physiologically compatible, water-soluble salts, 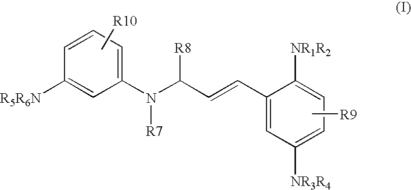 as well as dyes for keratin fibers that contain these compounds.

### FIELD OF THE INVENTION

The invention relates to novel p-diaminobenzene derivatives as well as agents containing these compounds for the coloring of keratin fibers.

### BACKGROUND OF THE INVENTION

In the area of coloring of keratin fibers, especially the coloring of hair, oxidative dyes have acquired a substantial significance. Coloring takes place through the reaction of certain developer substances with certain coupler substances in the presence of a suitable oxidizing agent. Specific examples of developer substances that can be employed include 2,5-diaminotoluene, 2,5-diaminophenylethanol, p-aminophenol, 4-amino-3-methylphenol and 1,4-diaminobenzene, while examples of coupler substances that can be named include resorcinol, 4-chlororesorcinol, 1-naphthol, 3-aminophenol, 5-amino-2-methylphenol and derivatives of m-phenylendiamine.

Furthermore, dyes that contain p-phenylendiamines substituted in the 2-position as the developer substance are described in DE-OS 100 14 855.

Regarding the oxidative dyes that are used in the coloring of human hair, there are numerous requirements imposed, in addition to a desired intensity. Thus, the dye must be safe from a toxicological and dermatological perspective, and the hair coloring obtained must exhibit good lightfastness, permanent wave fastness, acid fastness, and resistance to rubbing. In any case, such coloring must remain stable to the influence of light, rubbing, and chemical agents over a period of at least 4-6 weeks. An additional requirement is that it should be possible to produce a broad palette of different color shades by combining suitable developer substances and coupler substances.

However, it is not possible for the dyes currently employed to fulfill all of the aforementioned requirements.

There is thus a need for novel developer substances that substantially fulfill the aforementioned requirements.

### SUMMARY OF THE INVENTION

Unexpectedly, it has now been found that novel p-diaminobenzene derivatives of the general Formula (I) fulfill the established requirements for developer substances to a substantially high degree.

Thus, when these developer substances are used with known coupler substances, deep color shades are obtained that are extraordinarily, lightfast and resistant to washing.

The objects of the present invention are thus p-diaminobenzene derivatives of the general Formula (I) or their physiologically compatible, water-soluble salts,

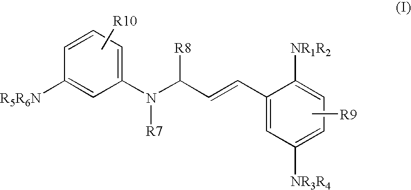

wherein

R1, R2, R3, R4, R5, R6, and R7 independently from one another stand for hydrogen, a C₁-C₆ alkyl group, a C₂-C₄ hydroxyalkyl group, a C₃-C₄ dihydroxyalkyl group or a C₁-C₄-alkoxy-(C₁-C₄)-alkyl group, or R1 and R2, R3 and R4, or R5 and R6 together with the N-atom can form a 4-membered to 8-membered aliphatic ring, provided that at least two of the groups R1 to R7 stand for hydrogen;

R8 equals a hydrogen or a C₁-C₄ alkyl group;

R9 and R10 independently from one another equal hydrogen, a halogen atom, a C₁-C₄ alkyl group, a C₁-C₄ hydroxyalkyl group, a C₁-C₄ hydroxyalkoxy group or a C₁-C₄ alkoxy group.

Examples of compounds of Formula (I) that can be named include:
3-[3-(2,5-diaminophenyl)allylamino]aniline; 3-[3-(2,5-diaminophenyl)allylamino)-6-(2-hydroxy-ethoxy)aniline; 3-[3-(2,5-diaminophenyl)allylamino)-6-fluoroaniline; 3-[3-(2,5-diamino-phenyl)allylamino)-6-chloroaniline; 3-[3-(2,5-diaminophenyl)allylamino)-6-methylaniline; 3-[3-(2,5-diaminophenyl)allylamino)4-chloroaniline; 3-[3-(2,5-diaminophenyl)allylamino)-4-methylaniline; 3-[3-(2,5-diaminophenyl)allylamino)-4-(2-hydroxyethoxy)aniline; 3-[3-(2,5-diaminophenyl)allylamino)-4-fluoroaniline; 3-[3-(2,5-diamino-3-(2-hydroxyethyl)phenyl)allyl-amino)aniline; 3-[3-(2,5-diamino-3-chlorophenyl)allylamino)aniline; 4-[3-(2,5-diamino-3-methyl-phenyl)allyl-amino)aniline; 3-[3-(2,5-diamino-6-methylphenyl)allylamino)aniline; 3-[3-(2,5-diamino-4-(2-hydroxyethyl)phenyl)allylamino)aniline; 3-[3-(2,5-diamino-4-chlorophenyl)-allylamino)aniline; 3-[3-(2,5-diamino-4-methylphenyl)allylamino)aniline; 3-[3-(N²,N²-bis-methyl-2,5-diaminophenyl)allylamino)aniline; 3-[3-(N²,N²-bis-(2-hydroxyethyl)-2,5-diaminophenyl)-allylamino)aniline; 3-[3-(N⁵,N⁵-bis-methyl-2,5-diaminophenyl)allylamino)-aniline; 3-[3-(N⁵,N⁵-bis-(2-hydroxyethyl)-2,5-diaminophenyl)allylamino)aniline, 3-[3-(2,5-diamino-phenyl)ally-lamino)-N,N′-bis-methylaniline; 3-[3-(2,5-diaminophenyl)allylamino)-N,N′-bis-(2-hydroxyethyl)-aniline or their physiologically compatible salts.

Preferred compounds of Formula (I) are those in which (i) at least one of the groups R8 to R10 stands for hydrogen and/or (ii) R1 and R2, R3 and R4, or R5 and R6, or all of the groups R1 to R4 or R1 to R7 stand for hydrogen. Further preferred compounds of Formula (I) are those in which R8 equals hydrogen and R5 and R6 independently from one another equal hydrogen, a C₁-C₄ alkyl group, a C₂-4 hydroxyalkyl group or a C₃-C₄ dihydroxyalkyl group.

Especially preferred p-diaminobenzene derivatives of Formula (I) are 3-[3-(2,5-diaminophenyl)allylamino]aniline, 3-[3-(2,5-diaminophenyl)allylamino)-6-(2-hydroxyethoxy)-aniline, 3-[3-(2,5-diaminophenyl)allylamino]-6-methylaniline and 3-[3-(2,5-diaminophenyl)-allylamino]-6-fluoro-aniline or their physiologically compatible salts.

The compounds of Formula (I) can be used either as the free bases or in the form of their physiologically compatible salts with inorganic or organic acids such as, for example, hydrochloric acid, sulfuric acid, phosphoric acid, acetic acid, propionic acid, lactic acid or citric acid.

The p-diaminobenzene derivatives of Formula (I) of the present invention can be prepared through the application of known synthesis routes, for example according to the following procedures.

Through a reductive amination of a substituted benzene of Formula (II) with an amine of Formula (III) and subsequent cleavage of the protecting group;

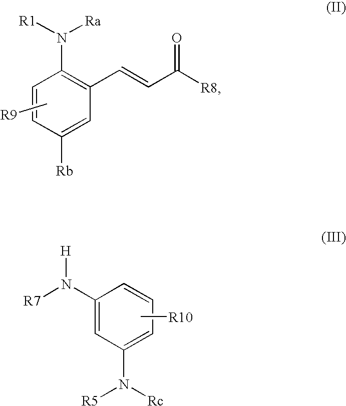

where Ra stands for a suitable protecting group, such as is described for example in Organic Synthesis, Chapter 7, “Protection for the Amino Group”, p 309, Wiley Interscience, 1991;

Rb means NR1Ra or NR1R2;

Rc means Ra or R6;

and R1, R2, R5, R6, R7, R8, R9, and R10 have the meanings specified in Formula (I).

The p-diaminobenzene derivatives of Formula (I) of the present invention are readily soluble in water and enable coloring with higher color intensity and excellent color fastness, especially as relates to lightfastness, resistance to washing, and resistance to rubbing. The compounds of Formula (I) furthermore exhibit excellent storage stability, especially as a component of the dyes described below.

Further objects of the present invention are thus compositions for the oxidative coloring of keratin fibers, such as for example hair, fur, feathers, or wool, especially human hair, based on a developer substance-coupler substance combination, which contains at least one p-diaminobenzene derivative of Formula (I) as a developer substance.

The p-diaminobenzene derivatives of Formula (I) are contained in the dyes of the present invention in total quantities of from about 0.005 through 20 percent by weight, where a quantity of from about 0.01 to 10 percent by weight is preferred, and of from 0.1 to 5 percent by weight is especially preferred.

Coupler substances that can be considered include preferably 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxy-ethoxy)-5-methylbenzene, 2,4-di-[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diamino-benzene, 2,4-diamino-1-(2-hydroxyethoxy)benzene, 2,4-diamino-1,5-di-(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di-(2-hydroxyethyl)amino]aniline, 4-amino-2-di-[(2-hydroxyethyl)amino]-1-ethoxybenzene, 5-methyl-2-(1-methylethyl)-phenol, 3-[(2-hydroxyethyl)amino]aniline, 3-[(2-aminoethyl)amino]aniline, 1,3-di-(2,4-diamino-phenoxy) propane, di-(2,4-diaminophenoxy)-methane, 1,3-diamino-2,4-dimethoxybenzene, 2,6-bis(2-hydroxyethyl)aminotoluene, 4-hydroxyindole, 3-dimethylaminophenol, 3-diethylamino-phenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxy-phenyl)amino]-acetamide, 5-[(2-hydroxyethyl)amino]-2-methylphenol, 3-[(2-hydroxy-ethyl)amino]phenol, 3-[(2-methoxyethyl)amino]phenol, 5-amino-2-ethylphenol, 2-(4-amino-2-hydroxy-phenoxy)ethanol, 5-[(3-hydroxypropyl)amino]-2-methylphenol, 3-[(2,3-dihydroxy-propyl)amino]-2-methylphenol, 3-[(2-hydroxyethyl)amino]-2-methylphenol, 2-amino-3-hydroxy-pyridine, 5-amino-4-chloro-2-methylphenol, 1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-di-hydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)-amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione.

Although the advantageous properties of the p-diaminobenzene derivatives of Formula (I) described here suggest their use singly as developer substances, it is obviously also possible to employ the p-diaminobenzene derivatives of Formula (I) together with known developer substances, such as for example 1,4-diaminobenzene, 2,5-diaminotoluene, 2,5-diaminophenylethanol, 4-aminophenol and their derivatives, for example 4-amino-3-methylphenol, 4,5-diaminopyrazole derivatives, for example 4,5-diamino-1-(2-hydroxyethyl)pyrazole, 4,5-diamino-1-isopropylpyrazole or 4,5-diamino-1-pentylpyrazole, or tetraaminopyrimidines.

The coupler substances and developer substances can be contained in the dyes of the present invention either singly or in mixtures with each other, where the total quantity of the coupler substances and developer substances in dyes of the present invention (based on the total quantity of the dye) amounts to from about 0.005 to 20 percent by weight, preferably from about 0.01 to 5.0 percent by weight, and especially from 0.1 to 2.5 percent by weight.

The total quantity of the developer substance-coupler substance combinations contained in the dyes described here amounts to from about 0.01 to 20 percent by weight, where an amount of from about 0.02 to 10 percent by weight is preferred, and 0.2 to 6 percent by weight is especially preferred. In general, the developer substances and coupler substances are employed in about equimolar amounts; it is however not disadvantageous if a certain excess or deficiency of the developer substances are present in this (for example, in a ratio of coupler substance to developer substance of from 1:2 to 1:0.5).

Furthermore, the dyes of the present invention can contain other color components in addition, for example 6-amino-2-methylphenol and 2-amino-5-methylphenol, as well as further conventional direct-penetrating dyes, for example triphenylmethane dyes such as 4-[(4′-aminophenyl)-(4′-imino-2″,5″-cyclohexadien-1″-ylidene)methyl]-2-methylaminobenzene mono-hydrochloride (C.I. 42 510) and 4-[(4′-amino-3′-methylphenyl)-(4″-imino-3″-methyl-2″,5″cyclohexadien-1″-ylidene)methyl]-2-methylaminobenzene monohydrochloride (C.I. 42 520), aromatic nitro dyes such as 4-(2′-hydroxyethyl)aminonitrotoluene, 2-amino-4,6-dinitrophenol, 2-amino-5-(2′-hydroxyethyl)aminonitrobenzene, 2-chloro-6-(ethylamino)-4-nitrophenol, 4-chloro-N-(2-hydroxyethyl)-2-nitroaniline, 5-chloro-2-hydroxy-4-nitroaniline, 2-amino-4-chloro-6-nitrophenol and 1-[(2′-ureidoethyl)amino-4-nitrobenzene, azo dyes as 6-[(4′-aminophenyl)azo]-5-hydroxynaphthalene-1-sulfonic acid sodium salt (C.I. 14 805) and dispersion dyes such as 1,4-diaminoanthraquinone and 1,4,5,8-tetraaminoanthraquinone.

The dyes of the present invention can contain these color components in an amount of from about 0.1 to 4 percent by weight.

Obviously, the coupler substances and developer substances as well as the other color components, insofar as they are bases, can also be employed in the in form of physiologically compatible salts with organic or inorganic acids, such as for example hydrochloric acid or sulfuric acid, or insofar as they possess aromatic OH groups, in the form of salts with bases, such as for example alkali phenolates.

Moreover, in the case that these dyes will be used in the coloring of hair, they can contain still further conventional cosmetic additives, for example antioxidants such as ascorbic acid, mercaptoacetic acid, or sodium sulfite, as well as perfume oils, chelating agents, wetting agents, emulsifiers, thickeners, and conditioning agents.

The formula of the dye of the present invention can be, for example, a solution, especially an aqueous or aqueous alcoholic solution. However, especially preferred formulations are a creme, a gel, or an emulsion. The composition of the solution represents a mixture of dye components with the conventional additives for such formulations.

Conventional additives in solutions, cremes, emulsions, or gels are, for example, solvents such as water, lower aliphatic alcohols, for example, ethanol, propanol or isopropanol, glycerin or glycols such as 1,2-propylenglycol, and furthermore, wetting agents or emulsifiers from the anionic, cationic, amphoteric, or nonionic classes of surface-active substances such as, for example, fatty alcohol sulfates, oxyethylated fatty alcohol sulfates, alkyl sulfonates, alkylbenzene sulfonates, alkyltrimethylammonium salts, alkyl betaines, oxyethylated fatty alcohols, oxyethylated nonylphenols, fatty acid alkanolamides and oxyethylated fatty acid esters, and moreover, thickeners such as higher fatty alcohols, starches, cellulose derivatives, petroleum jelly, paraffin oil, and fatty acids, and conditioning agents such as cationic resins, lanolin derivatives, cholesterol, pantothenic acid, and betaine. The components mentioned are used in the conventional amounts for such purposes, for example the wetting agents and emulsifiers are used in concentrations of from about 0.5 to 30 percent by weight, the thickeners in amounts of from about 0.1 to 25 percent by weight and the conditioning agents in a concentration of from about 0.1 to 5.0 percent by weight.

Depending on the composition, the dye of the present invention can be weakly acidic, neutral, or alkaline. In particular,the dyes of the present invention will exhibit a pH value of from 5 to 11.5, where a pH value of from about 6.5 to 10.5 is especially preferred. Adjustments to make the composition more basic are made preferably with ammonia, or can also be made with organic amines, for example monoethanolamine and triethanolamine, or inorganic bases such as sodium hydroxide and potassium hydroxide can be used. For pH-adjustments to make the composition more acidic, the use of inorganic or organic acids, for example phosphoric acid, acetic acid, citric acid, or tartaric acid, can be considered.

For applications in the oxidative coloring of hair, the aforedescribed dye is mixed with an oxidizing agent immediately before use, and an amount of this mixture that is sufficient for a hair coloring treatment, which in general is about 60 to 200 g (2.12 to 7.05 oz.) depending on the relative thickness of the hair, is applied to the hair.

Hydrogen peroxide or its addition compounds with urea, melamine, sodium borate, or sodium carbonate in the form of a from 3 to 12%, preferably 6%, aqueous solution, are primarily considered as suitable oxidizing agents for developing the hair coloring, but oxygen in the air can also be given consideration. If a 6% hydrogen peroxide solution is used as the oxidizing agent, the weight ratio between the hair dye and the oxidizing agent will be from 5:1 to 1:2, however preferably is 1:1. In particular, larger amounts of the oxidizing agent are used with higher dye concentrations in the hair dye, or if a stronger bleaching of the hair is desired at the same time. The mixture is allowed to react with the hair at from 15 to 50° C. for a period of about 10 to 45 minutes, and preferably for a period of 30 minutes, after which the hair is rinsed with water and dried. If necessary, this rinsing can be combined with a shampoo washing and with a possible post-rinse with a weak organic acid, such as for example citric acid or tartaric acid. Next the hair is dried.

The hair dye of the present invention that contains diaminobenzene derivatives of Formula (I) as a developer substance enables hair coloring with excellent color fastness, especially with regard to lightfastness, resistance to washing, and resistance to rubbing. With regard to the coloring properties, depending on the type and composition of the color components, hair dyes according to the present invention offer a broad palette of different color shades, which cover the range from blonde through brown, crimson, violet all the way to blue, and black color tones. The color tones here are noteworthy for their particular color intensity. The very good color properties of the hair dyes of the present application are further demonstrated by the fact that these agents enable the coloring of grayed hair that has had no prior chemical damage, in a problem-free manner with good coverage.

The following examples will illustrate the object of the invention without limiting the invention to these examples.

### EXAMPLES

### I. Examples of Preparations

### Example 1

### Synthesis of 3-[3-(2,5-diaminophenyl)allylamino]aniline Derivatives of Formula (I) (General Synthesis Procedure)

### A. Synthesis of 2,5-bis-tert-butyloxycarbonylaminobromobenzene

15.65 g of bromo-p-phenylendiamine hydrochloride and 32.7 g di-tert-butyl dicarbonate are dissolved in a mixture of 250 mL of 2N sodium hydroxide and 250 mL of trifluorotoluene, and this is warmed to 45° C.

The reaction mixture is stirred for 3 days. Thereafter, a total of 30 g of di-tert-butyl dicarbonate is added gradually. Next, the organic layer is separated and the aqueous phase is extracted twice with 100 mL of dichloromethane. The combined extracts are concentrated by evaporation and the residue is taken up in 200 mL of hexane. The precipitate is filtered off and is washed with 50 mL of hexane.

This yielded 18.6 g of 2,5-bis-tert-butyloxycarbonylaminobrombenzene with a melting point of 130° C.

### B. Synthesis of N-(4-tert-butyloxycarbonylamino-2-formylphenyl)carbamic acid tert-butyl ester

3.3 g of 2,5-bis-tert-butyloxycarbonylaminobromobenzene from step A is dissolved in 100 mL of anhydrous tetrahydrofuran under argon. Afterward, 17 mL of a 1.6 molar ethereal methyllithium solution is added gradually. The reaction mixture is cooled to −20° C. and then treated gradually with 7 mL of a 1.5 molar tert-butyllithium solution.

Once the addition is finished, the solution is stirred for a further 30 minutes at the specified temperature. Next, 1.2 g of dimethylformamide is added and the reaction mixture is stirred for 1 hour at −20° C. After warming slowly to room temperature, the reaction mixture is hydrolyzed with water and then poured into diethyl ether. The aqueous phase is then extracted with diethyl ether, and the organic phase is dried with magnesium sulfate. The solvent is distilled off on a rotary evaporator, and the residue is purified on silica gel with petroleum ether/ethyl acetate (9:1).

### C. Synthesis of N-(4-tert-butoxycarbonylamino-3-(3-oxopropenyl)phenyl)carbamic acid tert-butyl ester

9.5 g of N-(4-tert-butyloxycarbonylamino-2-formylphenyl)carbamic acid tert-butyl ester from step B is dissolved in 100 mL of tetrahydrofuran, and this is treated with 10.2 g of formylmethyltriphenylphosphorane. The reaction mixture is then stirred for 18 hours at 60° C. The reaction mixture is then poured into water, this is extracted with ethyl acetate, and the organic phase is washed with saturated aqueous NaCl solution, then dried with sodium sulfate, and concentrated after filtration.

Flash chromatography of the crude product on silica gel with hexane/EtOAc yields 4.84 g of N-(4-tert-butoxycarbonylamino-3-(3-oxopropenyl)phenyl)carbamic acid tert-butyl ester.

¹H-NMR (300 MHz, CDCl₃):

9.68 (d; J=7.6; 1H); 7.66 (d; J=2.2; 1H); 7.57 (d; J=15.9; 1H); 7.43 (d; J=8.6; 1H); 7.28 (dd; J¹=2.4; J²=8.7; 1H); 6.75 (s; 1H); 6.61 (dd; J²=7.6; J²=15.9; 1H); 6.51 (s; 1H); 1.51 (s; 18H)

### D. Synthesis of 3-[3-(2,5-diaminophenyl)allylamino]anilines

0.036 g of N-(4-tert-butoxycarbonylamino-3-(3-oxopropenyl)phenyl)carbamic acid tert-butyl ester from step C and 0.15 mmol of the corresponding amine are dissolved in 1,2-dichlorethane. Next, 0.1 mL of an acetic acid solution (1M in 1,2-dichlorethane) and 0.06 g of NaBH(OAc)₃ are added, and the reaction mixture is stirred for a period of from 5 to 15 hours at room temperature.

When the reaction is complete, the reaction mixture is poured into 10 mL of ethyl acetate, the organic phase is extracted with saturated sodium bicarbonate solution and then dried with magnesium sulfate. The solvent is distilled off on a rotary evaporator and the residue is purified on silica gel with petroleum ether/ethyl acetate (9:1). The product thus obtained is warmed in 4 mL of ethanol at 50° C. Then, the hydrochloride is prepared by dropwise addition of 1.5 mL of a 2.9 molar ethanolic hydrochloric acid solution. The precipitate is filtered off, and the solids are washed twice with 1 mL of ethanol and then dried.

### 1a. 3-[3-(2,5-Diaminophenyl)allylamino]aniline hydrochloride

Amine used: 3-tert-Butyloxycarbonylaminoaniline

Yield: 0.025 g

Mass spectrum: M⁺ 254 (20)

### 1b. 3-[3-(2,5-Diaminophenyl)allylamino]-6-(2-hydroxyethoxy)aniline hydrochloride and 3-[3-(2,5-diaminophenyl)allylamino]-4-(2-hydroxyethoxy)aniline hydrochloride

Amines used: 3-tert-Butyloxycarbonylamino-6-(2-hydroxyethoxy)aniline and 3-tert-butyloxycarbonylamino-4-(2-hydroxyethoxy)aniline, resp.

Yield: 0.025 g

Mass spectrum: M⁺ 328 (20)

### Examples 2 and 3

### Hair Dyes

Hair dye solution

| | | |
|---|---|---|
| 1.25 | mmol | Developer substance of Formula (I) according to |
| | | Table 1 |
| 1.25 | mmol | Coupler substance according to Table 1 |
| 1.0 | g | Potassium oleate (8% aqueous solution) |
| 1.0 | g | Ammonia (22% aqueous solution) |
| 1.0 | g | Ethanol |
| 0.3 | g | Ascorbic acid |

| | |
|---|---|
| balance to 100.0 g | Water |

50 g of the present dye solution is mixed with 50 g of a 6% aqueous hydrogen peroxide solution immediately before use. Then the mixture is applied to bleached hair. After an action period of 30 minutes at 40° C. the hair is rinsed with water, washed with a commercially available shampoo, and dried. The resulting colorings are compiled in Table 1.

TABLE 1

| | | Coupler substance | | | |
|---|---|---|---|---|---|
| | | | II. | | |
| | Developer | | 1,3-Diamino-4-(2- | | |
| | substance | I. | hydroxyethoxy)- | III. | |
| | of Formula | 1,3-Dihydroxy- | benzene sulfate | 5-Amino-2- | IV. |
| Example | (I) | benzene | Aminoanisol sulfate | methylphenol | 1-Naphthol |
| 2. | According to | Dark | Blue-gray | Crimson | Auburn |
| | Example 1a | blonde | | brown | |
| 3. | According to | Medium | Gray | Purple | Gray- |
| | Example 1b | blonde | | | violet |

### Examples 4 through 13

### Hair Dye

Hair dye solution

| X | g | 3-[3-(2,5-Diaminophenyl)allylamino]aniline |
|---|---|---|
| | | hydrochloride |
| | | (developer substance El of Formula (I)) |
| U | g | Developer substances E2 through E9 according |
| | | to the Table |
| Y | g | Coupler substances K12 through K33 according |
| | | to Table 3 |
| Z | g | 6-Chloro-2-ethylamino-4-nitrophenol (direct- |
| | | penetrating dye D2) |
| 10.000 | g | Potassium oleate (8% aqueous solution) |
| 10.000 | g | Ammonia (22% aqueous solution) |
| 10.000 | g | Ethanol |
| 0.300 | g | Ascorbic acid |

| | |
|---|---|
| balance to 100.000 g | Water |

30 g of the present dye solution is mixed with 30 g of a 6% aqueous hydrogen peroxide solution immediately before use. Then the mixture is applied to bleached hair. After an action period of 30 minutes at 40° C. the hair is rinsed with water, washed with a commercially available shampoo, and dried. The coloring results are compiled in Table 4.

### Examples 14 through 19

### Hair Dye

Creme-type color vehicle

| | | |
|---|---|---|
| X | g | 3-[3-(2,5-Diaminophenyl)allylamino]aniline hydro- |
| | | chloride (developer substance E1 of Formula (I)) |
| Y | g | Coupler substances K12 through K31 according to |
| | | Table 3 |
| Z | g | 6-Chloro-2-ethylamino-4-nitrophenol (direct- |
| | | penetrating dye D2) |
| 15.0 | g | Cetyl alcohol |
| 0.3 | g | Ascorbic acid |
| 3.5 | g | Sodium lauryl alcohol diglycol ether sulfate, 28% |
| | | aqueous solution |
| 3.0 | g | Ammonia 22% aqueous solution 0.3 g Sodium sulfite, |
| | | anhydrous |

| | |
|---|---|
| balance to 100 g | Water |

30 g of the present coloring cream was mixed with 30 g of a 6% hydrogen peroxide solution immediately before use. Then, the mixture is applied to the hair. After an action period of 30 minutes, the hair is rinsed with water, washed with a commercially available shampoo, and dried. The coloring results are compiled in Table 5.

TABLE 2
Developer substances

| | |
|---|---|
| E2 | 1,4-Diaminobenzene |
| E3 | 2,5-Diaminophenylethanol sulfate |
| E4 | 3-Methyl-4-aminophenol |
| E5 | 4-Amino-2-aminomethylphenol dihydrochloride |
| E6 | 4-Aminophenol |
| E8 | 4,5-Diamino-1-(2′-hydroxyethyl)pyrazole sulfate |
| E9 | 2,5-Diaminotoluene sulfate |

TABLE 3
Coupler substances

| | |
|---|---|
| K12 | 2-Amino-4-(2′-hydroxyethyl)aminoanisole sulfate |
| K13 | 1,3-Diamino-4-(2′-hydroxyethoxy)benzene sulfate |
| K21 | 3-Aminophenol |
| K22 | 5-Amino-2-methylphenol |
| K23 | 3-Amino-2-chloro-6-methylphenol |
| K25 | 1-Naphthol |
| K26 | 1-Acetoxy-2-methylnaphthalene |
| K31 | 1,3-Dihydroxybenzene |
| K32 | 2-Methyl-1,3-dihydroxybenzene |
| K33 | 1-Chloro-2,4-dihydroxybenzene |

TABLE 4
Hair dye

Example No.

| | | | |
|---|---|---|---|
| 4 | 5 | 6 | 7 |

| | |
|---|---|
| Dyes | (Quantity of dye in grams) |

| | | | | |
|---|---|---|---|---|
| E1 | 0.35 | 0.30 | 0.30 | 0.30 |
| E4 | 0.30 | | | |
| E5 | | 0.30 | | |
| E6 | | | 0.30 | |
| E8 | | | | 0.30 |
| K25 | 0.30 | 0.30 | | 0.30 |
| K26 | | | 0.35 | |
| K31 | 0.18 | | | 0.20 |
| K32 | | 0.22 | | |
| K33 | | | 0.20 | |
| Coloring results | Auburn | Auburn | Auburn | Auburn |

| | Example No. | | | | | |
|---|---|---|---|---|---|---|
| | 8 | 9 | 10 | 11 | 12 | 13 |
| Dyes | (Quantity of dye in grams) | | | | | |
| E1 | 0.50 | 0.40 | 0.40 | 0.16 | 0.15 | 0.15 |
| E2 | | | | 0.15 | | |
| E3 | | | | | 0.15 | |
| E9 | | | | | | 0.15 |
| K12 | | | 0.10 | | | |
| K13 | 0.09 | 0.09 | | | | |
| K21 | 0.05 | | | | | |
| K22 | | 0.05 | | | | |
| K23 | | | 0.05 | 0.10 | 0.10 | 0.10 |
| K31 | 0.20 | | | 0.15 | 0.20 | 0.10 |
| K32 | | 0.20 | | 0.10 | | 0.10 |
| K33 | | | 0.20 | | | |
| Coloring | Blonde | Blonde | Blonde | Blonde | Blonde | Blonde |
| results | | | | | | |

TABLE 5
Hair dye

| | Example No. | | | | | |
|---|---|---|---|---|---|---|
| | 14 | 15 | 16 | 17 | 18 | 19 |
| Dyes | (Quantity of dye in grams) | | | | | |
| E1 | 2.50 | 2.50 | 2.50 | 0.90 | 0.90 | 0.90 |
| K12 | | | | 0.10 | 0.10 | 0.10 |
| K13 | 1.10 | 1.10 | 1.10 | | | |
| K23 | | | 0.05 | 0.10 | 0.10 | 0.10 |
| K31 | 1.10 | 1.10 | 1.10 | 0.40 | 0.40 | 0.40 |
| D2 | | | | 0.10 | 0.10 | 0.10 |
| Coloring | Black | Black | Black | Brown | Brown | Brown |
| results | | | | | | |

Unless otherwise indicated, all percent values in the present application refer to percent by weight.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A p-Diaminobenzene derivative of the general Formula (I) or its physiologically compatible, water-soluble salts, 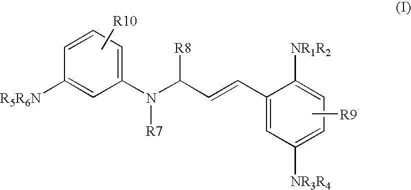
wherein
R1, R2, R3, R4, R5, R6, and R7 independently from one another are selected from hydrogen, a C₁-C₆ alkyl group, a C₂-C₄ hydroxyalkyl group, a
C₃-C₄ dihydroxyalkyl group or a C₁-C₄-alkoxy-(C₁-C₄)-alkyl group, or R₁ and R2, R3 and R4, or R5 and R6 together with the N atom form a 4-membered to 8-membered aliphatic ring, provided that at least two of the groups R1 to R7 are a hydrogen;
R8 equals hydrogen or a C₁-C₄ alkyl group;
R9 and R10 independently from one another equal hydrogen, a halogen atom, a C₁-C₄ alkyl group, a C₁-C₄ hydroxyalkyl group, a C₁-C₄ hydroxyalkoxy group or a C₁-C₄ alkoxy group.

2. A p-diaminobenzene derivative according to claim 1, wherein in Formula (I) (i) at least one of the groups R8 to R10 is hydrogen or (ii) R1 and R2, R3 and R4, R5 and R6, or all of the groups R1 to R4 or R1 to R7 are hydrogen.

3. A p-diaminobenzene derivative according to claim 1, wherein in Formula (I) R8 equals hydrogen and R5 and R6 independently from one another equal hydrogen, a C₁-C₄ alkyl group, a C₂-C₄ hydroxyalkyl group, or a C₃-C₄ dihydroxyalkyl group.

4. A p-diaminobenzene derivative according to claim 1, wherein said derivative is selected from the group comprising 3-[3-(2,5-diaminophenyl)allylamino]-aniline, 3-[3-(2,5-diaminophenyl)allylamino)-6-(2-hydroxyethoxy)aniline, 3-[3-(2,5-diaminophenyl)allylamino)-6-fluoroaniline, 3-[3-(2,5-diamino-phenyl)allylamino)-6-chloroaniline, 3-[3-(2,5-diaminophenyl)allylamino)-6-methylaniline, 3-[3-(2,5-diamino-phenyl)allylamino)-4-chloroaniline, 3-[3-(2,5-diaminophenyl)allylamino)-4-methyl-aniline, 3-[3-(2,5-diamino-phenyl)allylamino)-4-(2-hydroxyethoxy)aniline, 3-[3-(2,5-diaminophenyl)allylamino)-4-fluoroaniline, 3-[3-(2,5-diamino-3-(2-hydroxyethyl)-phenyl)allylamino)aniline, 3-[3-(2,5-diamino-3-chlorophenyl)allylamino)aniline, 4-[3-(2,5-diamino-3-methylphenyl)allylamino)aniline, 3-[3-(2,5-diamino-6-methylphenyl)allylamino)aniline, 3-[3-(2,5-diamino-4-(2-hydroxyethyl)phenyl)allylamino)-aniline, 3-[3-(2,5-diamino-4-chlorophenyl)allylamino)aniline, 3-[3-(2,5-diamino-4-methylphenyl)-allylamino)aniline, 3-[3-(N²,N²-bis-methyl-2,5-diaminophenyl)allylamino)aniline, 3-[3-(N²,N²-bis-(2-hydroxyethyl)-2,5-diaminophenyl)allylamino)aniline, 3-[3-(N⁵,N⁵-bis-methyl-2,5-diaminophenyl)allylamino)aniline,3-[3-(N⁵,N⁵-bis-(2-hydroxyethyl)-2,5-diaminophenyl)allylamino)aniline, 3-[3-(2,5-diaminophenyl)-allylamino)-N,N′-bis-methylaniline, 3-[3-(2,5-diaminophenyl)allylamino)-N,N′-bis-(2-hydroxyethyl)aniline, and the physiologically compatible salts thereof.

5. An agent for the oxidative coloring of keratin fibers based on a developer substance-coupler substance combination, wherein said agent comprises at least one p-diaminobenzene derivative of Formula (I) as recited in claim 1.

6. An agent according to claim 5 comprising the diaminobenzene derivative of Formula (I) in a quantity of from 0.005 to 20% by weight of said agent.

7. An agent according to claim 5, wherein the coupler substance is selected from the group consisting of 2,6-diaminopyridine, 2-amino-4-[(2-hydroxyethyl)amino]-anisole, 2,4-diamino-1-fluoro-5-methylbenzene, 2,4-diamino-1-methoxy-5-methyl-benzene, 2,4-diamino-1-ethoxy-5-methylbenzene, 2,4-diamino-1-(2-hydroxyethoxy)-5-methylbenzene, 2,4-di-[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)-benzene, 2,4-diamino-1,5-di-(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di-(2-hydroxyethyl)amino]aniline, 4-amino-2-di-[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di-(2,4-diaminophenoxy)propane, di-(2,4-diamino-phenoxy)methane, 1,3-diamino-2,4-dimethoxybenzene, 2,6-bis-(2-hydroxyethyl)aminotoluene, 4-hydroxyindole, 3-dimethylaminophenol, 3-diethyl-aminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichloro-phenol, 5-amino-2,4-dichlorophenol, 3-amino-2-methylphenol, 3-amino-2-chloro-6-methylphenol, 3-aminophenol, 2-[(3-hydroxy-phenyl)amino]acetamide, 5-[(2-hydroxy-ethyl)amino]-2-methyl-phenol, 3-[(2-hydroxyethyl)amino]phenol, 3-[(2-methoxyethyl)amino]phenol, 5-amino-2-ethylphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 1,5-dihydroxynaphthalene, 1,7-dihydroxy-naphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxy-benzene, 1,2-dichloro-3,5-dihydroxy-4-methylbenzene, 1,5-dichloro-2,4-dihydroxy-benzene, 1,3-dihydroxy-2-methylbenzene, 3,4-methylenedioxyphenol, 3,4-methylene-dioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-di-hydroxyindole, 5,6-dihydroxyindoline, 5-hydroxyindole, 6-hydroxyindole, 7-hydroxy-indole, and 2,3-indolinedione.

8. An agent according to claim 5, wherein said agent further comprises at least one additional developer substance, which is selected from the group consisting of 1,4-diaminobenzene, 2,5-diaminotoluene, 2,5-diaminophenylethanol, 4-aminophenol, 4-aminophenol derivatives, 4,5-diaminopyrazole derivatives and tetraaminopyrimidines.

9. An agent according to claim 5, comprising the developer substances and coupler substances in a total quantity of 0.005 to 20% by weight of said agent.

10. The agent according to claim 5, wherein said agent further comprises a direct-penetrating dye.

11. The agent according to claim 5, wherein said agent is a hair dye.
